(19) United States
(12) Patent Application Publication
(10) Pub. No.: US 2024/0366143 A1
(43) Pub. Date: Nov. 7, 2024

(54) METHOD AND SYSTEM FOR MONITORING PATIENTS FOR SEPSIS FOLLOWING DISCHARGE

(71) Applicant: KONINKLIJKE PHILIPS N.V., EINDHOVEN (NL)

(72) Inventors: CHAITANYA KULKARNI, BANGALORE (IN); DAVID PAUL NOREN, SHARON, MA (US); ERAN SIMHON, BOSTON, MA (US); VIKRAM SHIVANNA, BANGALORE (IN); LASITH ADHIKARI, REVERE, MA (US); GREGORY BOVERMAN, READING, MA (US); SYAMANTHAKA BALAKRISHNAN, BANGALORE (IN); LARRY JAMES ESHELMAN, OSSINING, NY (US)

(21) Appl. No.: 18/682,972

(22) PCT Filed: Aug. 4, 2022
(86) PCT No.: PCT/EP2022/071936

§ 371 Date: Feb. 12, 2024

Related U.S. Application Data
(60) Provisional application No. 63/232,247, filed on Aug. 12, 2021.

(51) Int. Cl. A61B 5/00; A61B 5/01; A61B 5/0205; A61B 5/021; A61B 5/024; A61B 5/08; A61B 5/28; G16H 10/20; G16H 10/60; G16H 15/00; G16H 20/10

(52) U.S. Cl. CPC A61B 5/412; A61B 5/02055; A61B 5/7267; A61B 5/7275; G16H 10/20; G16H 10/60; G16H 15/00; G16H 20/10; A61B 5/01; A61B 5/021; A61B 5/02416; A61B 5/0816; A61B 5/28

(57) ABSTRACT

A method (100) for predicting a sepsis status of a patient using a sepsis analysis system (200) comprising: (i) receiving (120) demographic information about the patient; (ii) receiving (130) observation data from a patient monitoring device; (iii) receiving (140) one or more patient responses provided by the patient in response to one or more queries or commands posed to the patient by the sepsis analysis system, and wherein the one or more queries or commands are configured to elicit, in the one or more patient responses, information relevant to the patient's sepsis status; (iv) analyzing (150), by a trained patient sepsis status model of the sepsis analysis system, the received demographic information, the received observation data, and the one or more patient responses to predict a sepsis status for the patient; and (v) reporting (170), via a user interface of the sepsis analysis system, the predicted sepsis status.

### FIELD OF THE DISCLOSURE

The present disclosure is directed generally to methods and systems for predicting a sepsis status of a patient post-discharge using a sepsis analysis system.

### BACKGROUND

Sepsis is a life-threatening illness which, without early intervention, can turn into septic shock which can comprise a mortality rate of 40%. Sepsis is caused by the body's overwhelming response to infection, and usually occurs in three stages. In the first stage, the infection reaches the blood stream and causes inflammation. In the second stage, the infection reaches other organs. The final stage is septic shock, which leads to organ failure. Sepsis treated at the early stage has a very high survival rate. The stage at which the patient is diagnosed is crucial and each stage has a different mortality rate. For example, there is a very high mortality rate within 28 days of severe sepsis and septic shock.

The main causes of sepsis are usually pneumonia, urinary tract infections, infections of the digestive system, and bloodstream infections. Septic infections are more common in the very old and the very young, as well as in people with a compromised immune system or a pre-existing condition such as diabetes. Sepsis is also common with patients who go through a major surgery or a procedure, which compromises the patient's immune system. As per the CDC, sepsis is diagnosed using four physical findings: (1) fever: (2) low blood pressure: (3) increased heart rate; and (4) difficulty breathing. While additional lab tests may be needed to confirm sepsis, these physical measurements assist in providing an initial indicator to recommend intervention. Since approximately 80% of sepsis begins outside of the hospital setting, intervention at the right time can save many lives.

Once a patient is discharged after a hospital stay, a patient is monitored for the next few days or weeks depending on the patient's needs for any suspicion of infection or sepsis. However, this monitoring depends on sporadic, if any, follow-up with the patient, as well as relying entirely on information self-reported by the patient. The patient may not be aware of the symptoms of sepsis, and may not answer questions truthfully.

### SUMMARY OF THE DISCLOSURE

There is a continued need for methods and systems that predict the sepsis status of a post-discharge patient. Accordingly, various embodiments and implementations herein are directed to methods and systems that analyze data from a post-discharge patient using a sepsis analysis system to more accurately predict the sepsis status of the patient. The system receives, from a patient database, demographic information about the patient. The system also receives observation data from a patient monitoring device. The system further receives one or more patient responses which are provided by the patient in response to one or more queries or commands posed to the patient by the sepsis analysis system. These queries or commands are configured to elicit, in the one or more patient responses, information relevant to the patient's sepsis status. Using the received demographic information, the received observation data, and the one or more patient responses, a trained patient sepsis status model of the sepsis analysis system predicts a sepsis status for the patient. The system reports the predicted patient sepsis status via a user interface of the sepsis analysis system.

Generally in one aspect, a method for predicting a sepsis status of a patient using a sepsis analysis system is provided. The method includes: (i) receiving, from a patient database, demographic information about the patient: (ii) receiving, at the sepsis analysis system, observation data from a patient monitoring device: (iii) receiving one or more patient responses, wherein the one or more patient responses are provided by the patient in response to one or more queries or commands posed to the patient by the sepsis analysis system, and wherein the one or more queries or commands are configured to elicit, in the one or more patient responses, information relevant to the patient's sepsis status: (iv) analyzing, by a trained patient sepsis status model of the sepsis analysis system, the received demographic information, the received observation data, and the one or more patient responses to predict a sepsis status for the patient; and (v) reporting, via a user interface of the sepsis analysis system, the predicted sepsis status for the patient.

According to an embodiment, the received observation data and the one or more patient responses to predict a sepsis status for the patient are received from a post-discharge patient.

According to an embodiment, the patient monitoring device is a wearable device.

According to an embodiment, the demographic information comprises one or more of: (i) dietary information, (ii) diagnosis information, and (iii) electronic medical record information, and the observation data comprises one or more of the patient's heart rate, an ECG, blood pressure, respiration rate, PPG, and temperature.

According to an embodiment, the one or more queries or commands comprise a request for symptom information from the patient.

According to an embodiment, the method further comprises the step of analyzing the received observation data to identify any data that may indicate possible patient sepsis.

According to an embodiment, the method further comprises the step of generating one or more queries or commands to pose to the patient, wherein the one or more queries or commands are generated based at least in part on the received observation data.

According to an embodiment, the one or more patient responses are received from the patient monitoring device or from a patient smartphone.

According to an embodiment, the one or more patient responses are analysed for patient sentiment.

According to an embodiment, the method further comprises the step of storing in a patient profile and/or patient database, some or all of the received observation data and the one or more patient responses.

According to a second aspect is a system configured to predicting a sepsis status of a patient. The system includes: a trained patient sepsis status model configured to generate a sepsis status for the patient based on received demographic information, received observation data, and one or more patient responses: a processor configured to: (i) receive, from a patient database, demographic information about the patient: (ii) receive, at the sepsis analysis system, observation data from a patient monitoring device; (iii) receive one or more patient responses, wherein the one or more patient responses are provided by the patient in response to one or more queries or commands posed to the patient by the sepsis analysis system, and wherein the one or more queries or commands are configured to elicit, in the one or more patient responses, information relevant to the patient's sepsis status: (iv) analyze, by the trained patient sepsis status model, the received demographic information, the received observation data, and the one or more patient responses to predict a sepsis status for the patient; and a user interface configured to provide the predicted sepsis status for the patient.

According to an embodiment, the received observation data and the one or more patient responses to predict a sepsis status for the patient are received from a post-discharge patient.

According to an embodiment, the processor is further configured to analyze the received observation data to identify any data that may indicate possible patient sepsis.

According to an embodiment, the processor is further configured to generate one or more queries or commands to pose to the patient, wherein the one or more queries or commands are generated based at least in part on the received observation data.

According to an embodiment, the processor is further configured to store in a patient profile and/or patient database, some or all of the received observation data and the one or more patient responses.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for predicting a sepsis status of a patient, in accordance with an embodiment.

FIG. 2 is a schematic representation of a sepsis analysis system, in accordance with an embodiment.

FIG. 3 is a flowchart of a method for training a sepsis status model, in accordance with an embodiment.

FIG. 4 is a flowchart of a method for training a sepsis status model, in accordance with an embodiment.

### DETAILED DESCRIPTION OF EMBODIMENTS

The present disclosure describes various embodiments of a system and method for predicting the sepsis status of a patient. More generally, Applicant has recognized and appreciated that it would be beneficial to provide a system configured to diagnose sepsis at an early stage in post-discharge patients. The system receives, from a patient database, demographic information about the patient. The system also receives observation data from a patient monitoring device. The system further receives one or more patient responses which are provided by the patient in response to one or more queries or commands posed to the patient by the sepsis analysis system. These queries or commands are configured to elicit, in the one or more patient responses, information relevant to the patient's sepsis status. Using the received demographic information, the received observation data, and the one or more patient responses, a trained patient sepsis status model of the sepsis analysis system predicts a sepsis status for the patient. The system reports the predicted patient sepsis status via a user interface of the sepsis analysis system.

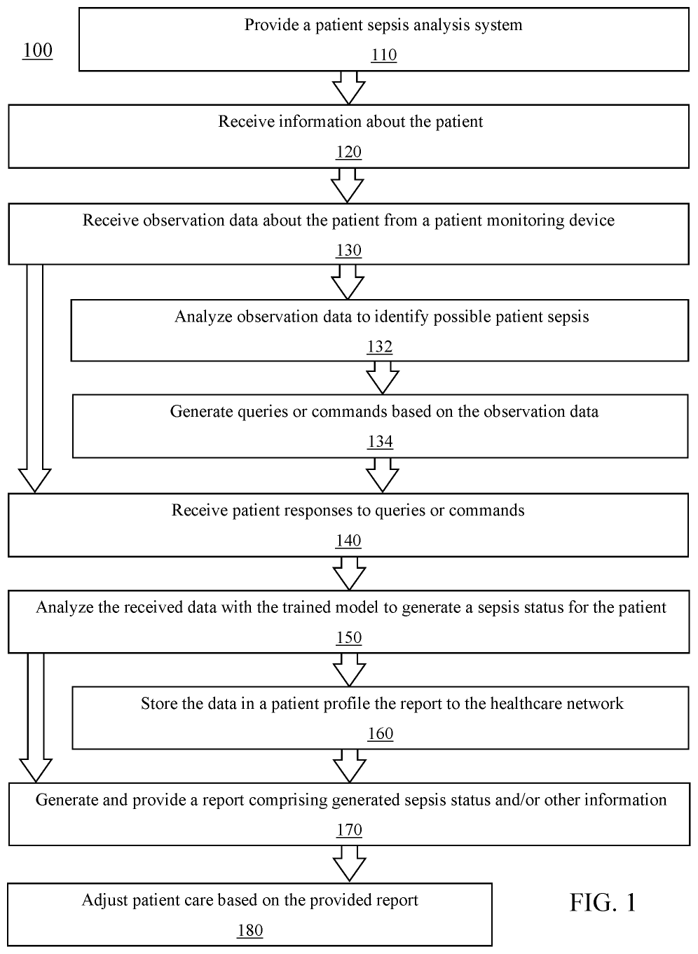

Referring to FIG. 1, in one embodiment, is a flowchart of a method 100 for predicting a sepsis status of a patient using a sepsis analysis system. The methods described in connection with the figures are provided as examples only, and shall be understood not limit the scope of the disclosure. The sepsis analysis system can be any of the systems described or otherwise envisioned herein and may comprise any of the components described or otherwise envisioned herein. The sepsis analysis system can be a single system or multiple different systems.

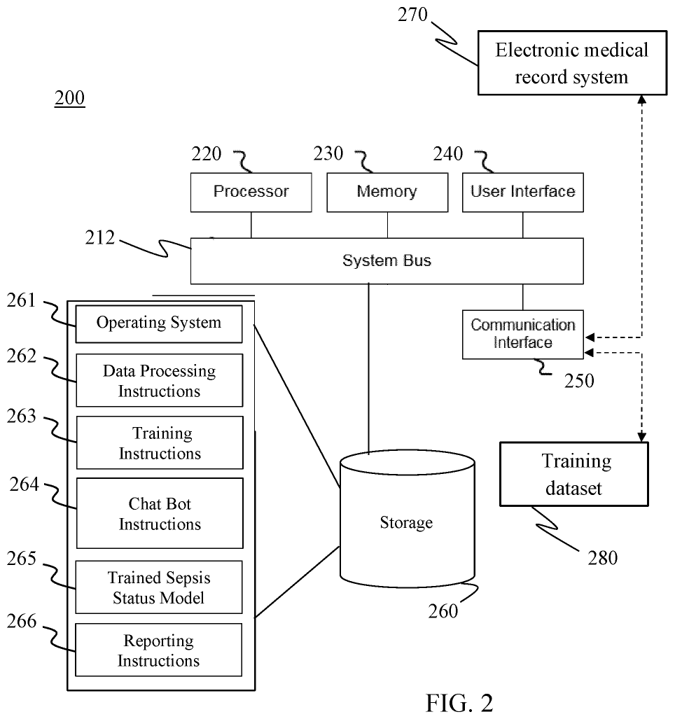

At step 110 of the method, a sepsis analysis system is provided. Referring to an embodiment of a sepsis analysis system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, sepsis analysis system 200 can be any of the systems described or otherwise envisioned herein. Other elements and components of sepsis analysis system 200 are disclosed and/or envisioned elsewhere herein.

At step 120 of the method, the sepsis analysis system receives information about a patient for which an analysis will be performed, According to an embodiment, the information comprises a plurality of features about the patient, Only some, or all, of the information about the patient and features may ultimately be utilized by the sepsis analysis system. The information about the patient and plurality of features may comprise, for example, demographic, dietary, and/or medical features about the patient. For example, the demographic, dietary, and/or medical information or features may comprise information such as patient preexisting conditions, diagnosis, past healthcare facility visits or admissions historical treatment, reasons for previous hospital visits, age, gender, height, weight, comorbidities, dietary information, and other demographic information. Medical information or features may comprise vital sign information about the patient, including but not limited to physiologic vital signs, physiological measurements other than vital data such as physical observations, patient diagnosis or medication condition, and more, among many other types of medical information.

According to an embodiment, reasons for previous hospital visits are used as features in the model. From the diagnosis of the patients, treatment diagnosis (such as the major diagnosis for the hospital visit) and comorbidities are extracted. The medication prescribed for the patient can also be used as inputs to the model. Many other types of patient information are possible. Accordingly, the received information can be any information relevant to a patient sepsis analysis or prediction.

The sepsis analysis system can receive patient information from a variety of different sources, including any source that comprises one or more patient features. According to an embodiment, the sepsis analysis system is in communication with an electronic medical records database from which the patient information and one or more of the plurality of features may be obtained or received. The electronic medical records database may be a local or remote database and is in communication with the sepsis analysis system 200. According to an embodiment, the sepsis analysis system comprises an electronic medical record database or system 270 which is optionally in direct and/or indirect communication with system 200. According to another embodiment, the sepsis analysis system may obtain or receive the information from equipment or a healthcare professional obtaining that information directly from the patient. According to another embodiment, the sepsis analysis system may obtain or receive the plurality of features from home monitoring equipment, such as a wearable device or any other device utilized by a patient outside of a hospital or other direct healthcare facility. According to an embodiment, the sepsis analysis system may query an electronic medical record database or system, comprising fast healthcare interoperability resources (FHIR) for example, to obtain the patient information.

The patient information received by the sepsis analysis system may be processed by the system according to methods for data handling and processing/preparation, including but not limited to the methods described or otherwise envisioned herein. The patient information received by the sepsis analysis system may be utilized, before or after processing, immediately or may be stored in local or remote storage for use in further steps of the method.

At step 130 of the method, the sepsis analysis system receives observation data from a patient monitoring device. The patient monitoring device can be any device configured to obtain observation data from a patient. According to an embodiment, the patient monitoring device is a wearable device such as a smartwatch, exercise monitoring device, harness, or other device. The patient monitoring device can be a handheld device such as a smartphone or other device. The patient monitoring device can be a household or IOT device in the patient's environment, such as household equipment, a smart mirror, or other devices. The patient monitoring device comprises one or more sensors configured to obtain observation data. The sensor may be, for example, an accelerometer, PPG sensor, heart rate sensor, respiratory sensor, heartbeat or rate sensor, thermometer, or other similar sensor, although many other sensors are possible. Accordingly, the patient monitoring device is in direct and/or indirect wired and/or wireless communication with the sepsis analysis system. The patient monitoring device can be configured to communicate observation data to the sepsis analysis system continually and/or periodically, and/or can communicate obtained observation data to the sepsis analysis system in response to a query from the sepsis analysis system.

As just one non-limiting example, the patient monitoring device can be a wearable device worn on the patient's wrist following discharge. The device comprises a pulse oximeter configured to obtain a photoplethysmogram (PPG) comprising a heart rate for the patient. The wearable device is connected to the internet directly, or connected to the patient's smartphone, and PPG data obtained over a previous 12-hour period is communicated to the sepsis analysis system via the internet. Many other devices, sensors, observation data, and time periods are possible.

According to an embodiment, a patient admitted to a hospital will be discharged, but upon discharge the patient may still be vulnerable to attacks from infections, especially following major surgery or chemotherapy. Since it is not possible to monitor patients for weeks following surgery at hospital, a home monitoring system can track the patient's health following discharge. For example, the doctor can recommend a monitoring window and can prescribe a wearables and a chat bot (described elsewhere herein) together as kit following discharge which can be used for monitoring the patient at home. Thus, on discharge, the patient can be prescribed a wearable/home monitoring kit and a chat bot and are instructed to record home monitoring and symptoms information at the appropriate time. For a sepsis patient, the home monitoring kit can contain a heart rate, ECG, BP, respiration, PPG (photoplethysmography) and/or temperature monitor for monitoring patient vitals. The wearable(s) can be connected to a cloud database to record all patient information. Since this information is acquired using a wearable, it can be collected at a high frequency. From the recorded vitals trend, features can be computed for data that was collected over the last 12 hours, 24 hours, 48 hours, or 96 hours, or any other time period. Trend features can include mean, median, variance, standard deviation and slope of all the features. Additional features such as ST elevation can be computed from an obtained ECG.

At step 140 of the method, the sepsis analysis system receives one or more patient responses. The one or more patient responses are provided by the patient in response to one or more queries or commands posed to the patient by the sepsis analysis system. The one or more queries or commands are configured to elicit, in the one or more patient responses, information relevant to the patient's sepsis status.

According to an embodiment, the system receives user feedback from a remote device such as a smartphone, computer, smart mirror, personal assistant device, or any other device capable of receiving a patient response to one or more queries or comments.

According to one embodiment, the system comprises a chat installed on the patient's phone, such as an app, configured to record real time symptoms and incidents. The chat bot can check on the patient for regular health. It can also request information from the patient to check if they are observing any symptoms of sepsis. The most common symptoms for sepsis are as follows as per the Mayo Clinic: (i) fever and chills: (ii) very low body temperature: (iii) infrequent urination: (iv) fast heartbeat: (v) nausea and vomiting: (vi) diarrhea: (vii) fatigue or weakness: (viii) blotchy or discolored skin: (ix) sweating or clammy skin; and/or (x) severe pain. Accordingly, the chat bot can be programmed to inquire about one or more of these symptoms. The chat bot can also inquire about the patient's activities of daily living (ADL behavior), among other information.

According to an embodiment, upon discharge, a patient can be prescribed with medications and with a custom diet based on their eating habits. As the diet and medications are linked to each patient, the chat bot can ask the patient with follow-up questions to record whether the prescribed diet plan and medications was followed. The chatbot application can also give reminders to the patient for food and medications consumption.

The chat bot conversation can also be used for sentiment analysis, for either text or voice reply. Sentiment can be mapped to a few features such as daily stress level or weakness level. These features can be used for improving the prediction performance.

According to an embodiment, at optional step 132 of the method the system can analyze received observation to identify any data that may indicate possible patient sepsis. For example, the system can be programmed or trained to identify data as being outside an acceptable range. The observation data can be compared to thresholds or can be classified to indicate possible patient sepsis. Further, at optional step 134 of the method, the system can generate one or more queries or commands to pose to the patient based at least in part on the received observation data.

For example, according to an embodiment, data on symptoms that cannot be monitored via the patient monitoring device, such as nausea or pain level can be collected by the chat bot on a regular basis, such as daily. The chat bot can be programed to ask a set of questions in a text or voice format. In addition, if the patient monitoring device fails to collect some of the data or if some of the data points are abnormal, the chat bot application can detect that data is missing/abnormal and can generate appropriate questions. For example, if at some point the patient monitoring device reports extremely high or low body temperature, then the chat bot can ask the patient to measure the patient's body temperature with a thermometer and to report the result manually. To detect abnormal results, an anomaly detection algorithm can periodically and/or constantly run in the background. Anomaly can be detected by known statistical tools such as Z-Score analysis or Extreme Value Analysis.

According to an embodiment, an algorithm for the identification of sepsis risk (i.e., sepsis risk index) can be run periodically and multiple times in a day, such as when patient vital information is updated. If the model consistently indicates a high risk over a period of time, the chat bot can ask questions to the patient through the app to increase the confidence of the risk. The patient may update the questionnaire with a copy of recorded vitals, previous medical history, dietary information, and/or medications, the risk from the sepsis risk index and the questionnaire is sent to the doctor in the form of a document and a tele conference is set up for the doctor to meet the patient and decide on the future course of action.

At step 150 of the method, a trained patient sepsis status model of the sepsis analysis system analyzes the received patient information, the received observation data, and the one or more patient responses to predict a sepsis status for the patient. The trained model can be any algorithm, module, or component configured to analyze input data to classify the input or otherwise generate or predict a sepsis status for the patient.

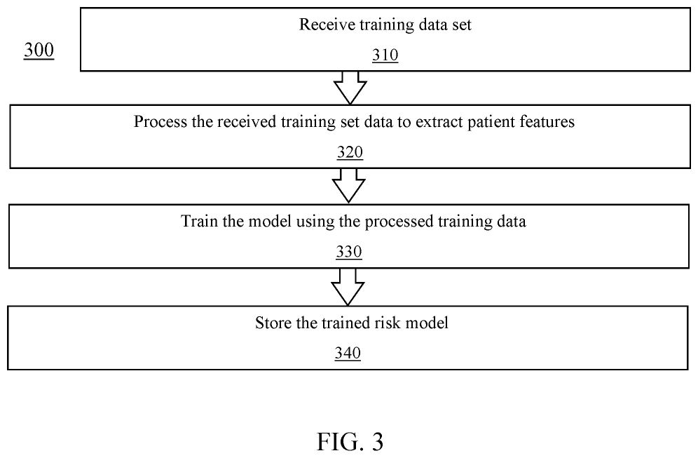

Referring to FIG. 3 in one embodiment, is a method 300 for training the patient sepsis status model of the sepsis analysis system. At step 310 of the method, the system receives a training data set comprising training data about a plurality of patients. The training data can comprise demographic, dietary, and/or medical features about the patient. For example, the demographic, dietary, and/or medical information or features may comprise information such as patient preexisting conditions, diagnosis, past healthcare facility visits or admissions historical treatment, reasons for previous hospital visits, age, gender, height, weight, comorbidities, dietary information, and other information. Medical information or features may comprise vital sign information about the patient, including but not limited to physiologic vital signs, physiological measurements other than vital data such as physical observations, patient diagnosis or medication condition, and more, among many other types of medical information. According to an embodiment, the training data may comprise retrospective admission/discharge/transfer (ADT) data. The training data may be stored in and/or received from one or more databases. The database may be a local and/or remote database. For example, the system may comprise a database of training data, such as database 280 in FIG. 2.

According to an embodiment, the sepsis analysis system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 320 of the method, the system processes the received information to extract sepsis prediction features about one or more of the plurality of patients. The sepsis prediction features may be any features which will be utilized to train the sepsis status model, such as any sepsis prediction features that can or will be utilized by the trained algorithm for sepsis risk analysis for a future patient. Feature extraction can be accomplished by a variety of embodiments for feature identification, extraction, and/or processing, including any method for extracting features from a dataset. The outcome of a feature processing step or module of the sepsis analysis system is a set of sepsis prediction features about a plurality of patients, which thus comprises a training data set that can be utilized to train the classifier.

At step 330 of the method, the system trains the machine learning algorithm, which will be the algorithm utilized in analyzing input information as described or otherwise envisioned. The machine learning algorithm is trained using the extracted features according to known methods for training a machine learning algorithm, According to an embodiment, the algorithm is trained, using the processed training dataset, to generate a sepsis status or prediction for a patient. According to an embodiment, the algorithm is also trained, using the processed training dataset, to generate one or more intervention recommendations. At step 340 of the method, the trained model is stored for future use. According to an embodiment, the model may be stored in local or remote storage.

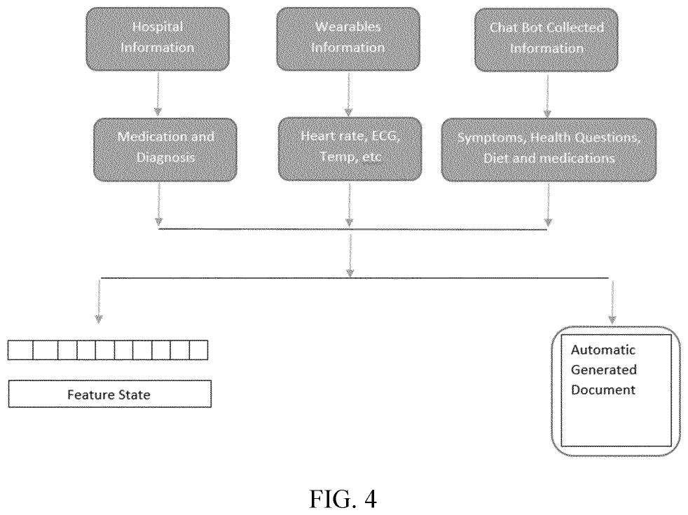

Referring to FIG. 4, in one embodiment, is a flowchart of a method for training the patient sepsis status model. According to an embodiment, the training data can be used to build documents automatically. Each of these documents can be annotated by trainers for three different classes such as the following: (i) no intervention needed: (ii) intervention needed; and (iii) readmission needed. Using these features and annotation, a classifier can be built to predict sepsis risk. A tree-based classifier such as random Forrest or XGBoost can be used to develop a classifier. Alternately, a neural network classifier can also be used. This model can be run on the patient at intervals such as every 2 hours, although many other intervals are possible to estimate the sepsis risk. Alternatively, a temporal model such as Recurrent neural network (RNN) can be used. Such a model will consider current status of the patient combined with historic data. The advantage of such model is that it can pick up trends and give higher weights to more recent data.

This model can be easily updated using a continuous feedback mechanism. In the feedback mechanism when the model generates a false positives or true negatives the doctor can look at the report the model generates and change its label. The updated labels are used to re-train the model. This will improve the model performance and will guarantee that the model stays relevant over time.

According to an embodiment, the model can be configured or trained to generate sepsis risk scores. If the patient is observed to have continuously high risk over a certain duration, the hospital will be notified. The hospital will also receive information about features contributing to the risk. The features contributing to the risk can be generated from comparing the old results (or baseline results which can be obtained before hospital discharge) where no risk was observed to the current readings. All home monitoring information will be structured with the patient's chat bot notes and sent to the hospital. Based on this information, a tele-health call will be scheduled with the patient.

At optional step 160 of the method in FIG. 1, the sepsis analysis system stores some or all of the received observation data and the one or more patient responses in a patient profile and/or patient database, database may be a local and/or remote database. For example, the system may comprise a database of patient profiles. A patient profile or patient document can be annotated based on the predicted or generated sepsis status with one or more of the following classes, among other possible classes or labels: (i) no intervention needed: (ii) intervention needed; and (iii) readmission needed.

At step 170 of the method in FIG. 1, the sepsis analysis system reports the predicted or generated sepsis status for the patient via a user interface. Alternatively, the information may be communicated by wired and/or wireless communication to another device. For example, the system may communicate the information to a mobile phone, computer, laptop, wearable device, and/or any other device configured to allow display and/or other communication of the information. For example, an intended recipient may be a healthcare professional that will make decisions or evaluate the sepsis status to impact the patient's care. In addition to the generated sepsis status for the patient, the report can comprise any other information such as information about the patient including the patient demographics, the received observation data, and the one or more patient responses to predict a sepsis status for the patient, among other possible information.

Accordingly, at step 180 of the method, the decision maker adjusts care for the patient in response to the reported predicted sepsis status for the patient, wherein adjusting care comprises a sepsis intervention. For example, the healthcare professional may direct the patient to check-in to a care facility if there is a high risk of sepsis. The healthcare professional may prescribe medication or make any other patient care decision in response to the predicted sepsis status. Step 180 of the method may include review or other consultation of the annotated patient profile or patient document, and making a care decision for the patient or adjusting patient care based on the annotated patient profile or document. For example, if the annotated patient profile or document is annotated with “intervention needed.” the decision maker such as a physician may prescribe antibiotics or another medication. As another example, if the annotated patient profile or document is annotated with “readmission needed, the decision maker such as a physician may direct the patient to check in at a care facility for monitoring and advanced intervention.

Referring to FIG. 2 is a schematic representation of a sepsis analysis system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, storage 260 of system 200 may store one or more algorithms, modules, and/or instructions to carry out one or more functions or steps of the methods described or otherwise envisioned herein. For example, the system may comprise, among other instructions or data, an electronic medical record system 270, training dataset 280, data processing instructions 262, training instructions 263, chat bot instructions 264, trained sepsis status model 265, and/or reporting instructions 266.

According to an embodiment, the electronic medical record system 270 is an electronic medical records database from which the information about the patient, including the plurality of leakage prediction features, may be obtained or received. The electronic medical records database may be a local or remote database and is in communication the sepsis analysis system 200. According to an embodiment, the sepsis analysis system comprises an electronic medical record database or system 270 which is optionally in direct and/or indirect communication with system 200.

According to an embodiment, the training data set 280 is a dataset that may be stored in a local or remote database and is in communication the sepsis analysis system 200. According to an embodiment, the sepsis analysis system comprises a training data set 280. The training data can comprise medical information about a patient, including but not limited to demographics, physiological measurements such as vital data, physical observations, and diagnosis, among many other types of information as described or otherwise envisioned herein.

According to an embodiment, data processing instructions 262 direct the system to retrieve and process input data which is used to train the patient sepsis status model 265. The data processing instructions 262 direct the system to, for example, receive or retrieve input data to be used by the system as needed, such as from electronic medical record system 270 among many other possible sources. As described above, the input data can comprise a wide variety of input types from a wide variety of sources, including home monitoring devices and other patient monitoring devices, among other devices and systems.

According to an embodiment, the data processing instructions 262 also direct the system to process the input data to generate a plurality of sepsis prediction features related to medical information for a plurality of patients, which are used to train the classifier. This can be accomplished by a variety of embodiments for feature identification, extraction, and/or processing. The outcome of the feature processing is a set of sepsis prediction features related to sepsis analysis for a patient, which thus comprises a training data set that can be utilized to train the model 265.

According to an embodiment, training instructions 263 direct the system to utilize the processed data to train the sepsis status model 265. The sepsis status model can be any machine learning algorithm, classifier, or model sufficient to utilize the type of input data provided, and to generate a patient sepsis status analysis. Thus, the system comprises a trained sepsis status model 265 configured to generate a sepsis analysis for a patient, as described or otherwise envisioned herein.

According to an embodiment, the chat bot instructions 264 direct the system to generate and/or provide one or more questions or commands to the patient. The one or more queries or commands are configured to elicit, in the one or more patient responses, information relevant to the patient's sepsis status, as described or otherwise envisioned herein. For example, the chat bot instructions 264 can direct the system to provide one or more queries or commands to the patient via a user device such as a smartphone, computer, monitoring device, or any other device.

According to an embodiment, reporting instructions 265 direct the sepsis analysis system to generate and provide to a user via a user interface information comprising a generated report comprising the sepsis status or prediction for a patient. In addition to the generated sepsis status for the patient, the report can comprise any other information such as information about the patient including the patient demographics, the received observation data, and the one or more patient responses to predict a sepsis status for the patient, among other possible information. The information may be communicated by wired and/or wireless communication to another device. For example, the system may communicate the information to a mobile phone, computer, laptop, wearable device, and/or any other device configured to allow display and/or other communication of the information.

According to an embodiment, the sepsis analysis system is configured to process many thousands or millions of datapoints in the input data used to train the classifier, as well as to process and analyze the received plurality of patient features. For example, generating a functional and skilled trained classifier using an automated process such as feature identification and extraction and subsequent training requires processing of millions of datapoints from input data and the generated features. This can require millions or billions of calculations to generate a novel trained classifier from those millions of datapoints and millions or billions of calculations. As a result, each trained classifier is novel and distinct based on the input data and parameters of the machine learning algorithm, and thus improves the functioning of the network leakage analysis system. Thus, generating a functional and skilled trained classifier comprises a process with a volume of calculation and analysis that a human brain cannot accomplish in a lifetime, or multiple lifetimes.

In addition, the sepsis analysis system can be configured to continually receive patient features, perform the analysis, and provide periodic or continual updates via the report provided to a user for the patient. This requires the analysis of thousands or millions of datapoints on a continual basis to optimize the reporting, requiring a volume of calculation and analysis that a human brain cannot accomplish in a lifetime. By providing an improved sepsis analysis or prediction, this novel network sepsis analysis system has an enormous positive effect on patient outcomes, including saving many lives.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of.” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising.” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method (100) for predicting a sepsis status of a patient using a sepsis analysis system (200) comprising:
receiving (120), from a patient database, demographic information about the patient;
receiving (130), at the sepsis analysis system, observation data from a patient monitoring device;
receiving (140) one or more patient responses, wherein the one or more patient responses are provided by the patient in response to one or more queries or commands posed to the patient by the sepsis analysis system, and wherein the one or more queries or commands are configured to elicit, in the one or more patient responses, information relevant to the patient's sepsis status;
analyzing (150), by a trained patient sepsis status model of the sepsis analysis system, the received demographic information, the received observation data, and the one or more patient responses to predict a sepsis status for the patient; and
reporting (170), via a user interface of the sepsis analysis system, the predicted sepsis status for the patient.

2. The method of claim 1, wherein the received observation data and the one or more patient responses to predict a sepsis status for the patient are received from a post-discharge patient.

3. The method of claim 1, wherein the patient monitoring device is a wearable device.

4. The method of claim 1, wherein the demographic information comprises one or more of: (i) dietary information, (ii) diagnosis information, and (iii) electronic medical record information, and wherein the observation data comprises one or more of the patient's heart rate, an ECG, blood pressure, respiration rate, PPG, and temperature.

5. The method of claim 1, wherein the one or more queries or commands comprise a request for symptom information from the patient.

6. The method of claim 1, further comprising the step of analyzing (132) the received observation data to identify any data that may indicate possible patient sepsis.

7. The method of claim 1, further comprising the step of generating (134) one or more queries or commands to pose to the patient, wherein the one or more queries or commands are generated based at least in part on the received observation data.

8. The method of claim 1, wherein the one or more patient responses are received from the patient monitoring device or from a patient smartphone.

9. The method of claim 1, wherein the one or more patient responses are analysed (142) for patient sentiment.

10. The method of claim 1, further comprising the step of storing (160) in a patient profile and/or patient database, some or all of the received observation data and the one or more patient responses.

11. A system (200) configured to predicting a sepsis status of a patient, comprising:
a trained patient sepsis status model (265) configured to generate a sepsis status for the patient based on received demographic information, received observation data, and one or more patient responses;
a processor (220) configured to: (i) receive, from a patient database, demographic information about the patient: (ii) receive, at the sepsis analysis system, observation data from a patient monitoring device: (iii) receive one or more patient responses, wherein the one or more patient responses are provided by the patient in response to one or more queries or commands posed to the patient by the sepsis analysis system, and wherein the one or more queries or commands are configured to elicit, in the one or more patient responses, information relevant to the patient's sepsis status: (iv) analyze, by the trained patient sepsis status model, the received demographic information, the received observation data, and the one or more patient responses to predict a sepsis status for the patient; and
a user interface (240) configured to provide the predicted sepsis status for the patient.

12. The system of claim 11, wherein the received observation data and the one or more patient responses to predict a sepsis status for the patient are received from a post-discharge patient.

13. The system of claim 11, wherein the processor is further configured to analyze the received observation data to identify any data that may indicate possible patient sepsis.

14. The system of claim 11, wherein the processor is further configured to generate one or more queries or commands to pose to the patient, wherein the one or more queries or commands are generated based at least in part on the received observation data.

15. The system of claim 11, wherein the processor is further configured to store in a patient profile and/or patient database, some or all of the received observation data and the one or more patient responses.
